SEQUENCE LISTING (9 sequences)

SEQ ID NO: 1 - protein, 123 aa, Porcine reproductive and respiratory syndrome virus
MPNNNGKQQKRKKGDGQPVNQLCQMLGKIIAQQNQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 2 - protein, 123 aa, Porcine reproductive and respiratory syndrome virus
MPNNNGKQQKRKKGDGQPVNQLCQMLGKTIAQQNQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 3 - protein, 123 aa, Porcine reproductive and respiratory syndrome virus
MPNNNGKQQKRKKGDGQPVNQLCQMLGKIIAQQNQSRGKGPGKKNKKKNPEKPDFPLATEDDVRHHFTRSERQLCLSSIQTAFNQGAGTCTLSDSGRISYTVEFSLPTHHTVRLIRVTASPSA

SEQ ID NO: 4 - protein, 123 aa, Porcine reproductive and respiratory syndrome virus
MPNNNGKQRKKKKGNGQPVNQLCQMLGKIIAQQNQSRGKGPGKKNKKKSPEKPDFPLATEDDVRHHFTRSERQLCLSSIQTAFNQGAGTCTLSDSGRISYTVEFSLPTHHTVRLIRVTASPSA

SEQ ID NO: 5 - protein, 123 aa, Porcine reproductive and respiratory syndrome virus
MPNNNGKQQKKKKGNGQPVNQLCQMLGKIIAQQNQSRGKGPGKKRKKKNPEKPHFPLATEDDVRHHFTPSERQLCLSSIQTAFNQGAGTCALSDSGRISYTVEFSLPTQHTVRLIRATASPSA

SEQ ID NO: 6 - DNA, 18 bp, Artificial sequence
  Synthetic oligonucleotide
gcggccgcactagtgata

SEQ ID NO: 7 - DNA, 19 bp, Artificial sequence
  Synthetic oligonucleotide
gatggagaagcggtaaccc

SEQ ID NO: 8 - DNA, 25 bp, Artificial sequence
  Synthetic oligonucleotide
gtgacttgaaccatctgatctcagc

SEQ ID NO: 9 - DNA, 22 bp, Artificial sequence
  Synthetic oligonucleotide
gttgaagccatcctcaagccag

(19) United States
(12) Patent Application Publication
(10) Pub. No.: US 2014/0096285 A1
(43) Pub. Date: Apr. 3, 2014

(54) SOYBEAN-BASED PORCINE REPRODUCTIVE AND RESPIRATORY SYNDROME VIRUS VACCINE AND METHODS FOR MAKING AND USING THE SAME

(71) Applicant: The Board of Trustees of the University of Illinois, Urbana, IL (US)

(72) Inventor: Schuyler S. Korban, Champaign, IL (US)

(73) Assignee: The Board of Trustees of the University of Illinois, Urbana, IL (US)

(21) Appl. No.: 14/044,464

(22) Filed: Oct. 2, 2013

Related U.S. Application Data
(60) Provisional application No. 61/708,771, filed on Oct. 2, 2012.

(51) Int. Cl. C07K 14/005; C12N 15/82

(52) U.S. Cl. CPC C07K 14/005; C12N 15/8243
USPC 800/294; 800/312; 435/320.1; 435/415; 530/350; 424/204.1

(57) ABSTRACT

A transgenic soybean plant harboring an exogenous nucleic acid molecule encoding porcine reproductive and respiratory syndrome virus ORF7 immunogen and methods for using the same to induce an antibody response and prevent a porcine reproductive and respiratory syndrome virus infection are provided.

### INTRODUCTION

This application claims the benefit of priority of U.S. Provisional Application No. 61/708,771, filed Oct. 2, 2012, the content of which is incorporated herein by reference in its entirety.

This invention was made with government support under contract number G012026-220 awarded by the Department of Energy. The government has certain rights in this invention.

### BACKGROUND

Porcine reproductive and respiratory syndrome (PRRS) is one of the most serious health problems in breeding herd. The causative agent of this disease is the PRRS virus, designated as PRRSV. The PRRS virus, an arterivirus, infects swine resulting in both respiratory and reproductive distress (Bernard, et al. (1996) Fields Virology. Lippincott-Raven Publishers, Philadelphia). Clinical symptoms vary from no symptoms to death observed in pigs of all age groups, but particularly pregnant sows, resulting in respiratory distress, pneumonia, and increasing pre-weaning mortality. PRRSV spreads rapidly and causes economic losses in many swine-producing countries. In addition to innovative diagnostic tools, efficient vaccines are much needed for PRRS disease control.

Currently, a modified-live virus (MLV) has been used to prevent infection against PRRSV; however, this does not serve as an effective vaccine as it has not been efficacious when applied in the field (Bouwkamp (1999) Tijdschr. Diergeneeskd. 124:530-535; Hurd, et al. (2001) J. Swine Health Product. 9:103-108). Alternative approaches have been proposed to develop vaccines against PRRS (see US 2003/0049274) and other mammalian diseases. Among those, subunit vaccines have been proposed, wherein only epitopes of the pathogen are used for vaccine development (Monger, et al. (2006) Plant Biotechnol. J. 4:623-631; Obregon, et al. (2006) Plant Biotechnol. J. 4:195-207; Nochi, et al. (2007) Proc. Natl. Acad. Sci. USA 104:10986-10991; Soria-Guerra, et al. (2010) Planta 229:1293-1302; Rosales-Mendoza, et al. (2011) Plant Cell Rep. 30:1145-52). Although subunit vaccines can be produced in bacterial or insect all cultures in large fermentors, plants have been proposed as alternative systems for production (Arntzen, et al. (2005) Vaccine 23:1753-1756; Twyman, et al. (2003) Trends Biotechnol. 21:570-578; Horn, et al. (2004) Plant Cell Rep. 22:711-720).

Plant-based oral vaccines against cattle (Dus Santos & Wigdorovitz (2005) Immunol. Cell Biol. 83:229-238; Floss, et al. (2007) Transgenic Res. 16:315-332) and swine diseases (Kang, et al. (2005) Vaccine 23:2294-2297; Oszvald, et al. (2007) Mol. Biotechnol. 35:215-223) have been described. See also U.S. Pat. No. 7,723,570. A candidate subunit vaccine against porcine transmissible gastroenteritis virus has been developed in transgenic corn (Streatfield, et al. (2001) Vaccine 21:812-815). Animal feeding studies have demonstrated that the corn-based porcine transmissible gastroenteritis virus candidate vaccine provides protection against infection by the virus (Lamphear, et al. (2002) J. Controlled Release 85:169-180). Furthermore, ORF5 of PRRSV, along with an LTB (heat-labile enterotoxin B subunit of Escherichia coli) as an adjuvant have been introduced into tobacco (Chia, et al. (2010) Vet. Immunol. Immunopathol. 135:234-242; Chia, et al. (2011) Vet. Immunol. Immunopathol. 140:215-225) and potato (Chen & Lu (2011) J. Virol. Meth. 173:153-158), and shown to stimulate both serum and gut mucosal-specific antibodies in mice. Pigs immunized with transgenic tobacco expressing ORF5 developed PRRSV-specific antibody- and cell-mediated immunity, and showed significantly lower viremia and tissue viral load and milder lung lesions than wild-type tobacco plant (Chia, et al. (2011) supra).

### SUMMARY OF THE INVENTION

The present invention is a transgenic soybean plant harboring an exogenous nucleic acid molecule encoding porcine reproductive and respiratory syndrome virus (PRRSV) ORF7 immunogen and methods for orally administering the same to induce an antibody response and prevent a porcine reproductive and respiratory syndrome virus infection.

The invention also provides an expression construct for expressing a nucleic acid molecule encoding PRRSV ORF7 immunogen, wherein in certain embodiments, the expression construct includes a hygromycin resistance gene, Agrobacterium tumifaciens T-DNA left and right border repeats, and a cauliflower mosaic virus 35S constitutive promoter and terminator flanking the PRRSV ORF7 nucleic acid molecule.

This invention further provides a method for preparing a PRRSV ORF7 immunogen in soybean by a modifying a PRRSV ORF7 nucleic acid for expression in soybean, inserting the modified PRRSV ORF7 nucleic acid into an expression construct, introducing the expression construct into Agrobacterium tumifaciens, and transforming soybean with the Agrobacterium tumifaciens.

As another embodiment, the invention includes a method for obtaining a purified PRRSV ORF7 protein by growing a transgenic soybean plant harboring an exogenous nucleic acid molecule encoding PRRSV ORF7 immunogen for a time sufficient to produce seeds containing the PRRSV ORF7 protein, harvesting the seeds from the transgenic soybean plant, wherein the PRRSV ORF7 protein constitutes at least 2.0% of the total soluble protein in the harvested seeds, and purifying the PRRSV ORF7 protein from the harvested seeds.

The present invention also provides a plant part and a seed from the transgenic soybean plant, wherein at least 2.0% of total soluble protein of the seed is the PRRSV ORF7 immunogen. A food product including the transgenic soybean plant, or seed or plant part thereof, is also provided.

### BRIEF DESCRIPTION OF THE DRAWINGS

FIG. 1 depicts an illustrative embodiment of the design of expression vectors. The binary vector pH2GW7 cassette comprises a hygromycin resistance gene (Hyg), A. tumefaciens T-DNA left and right border repeats (LB and RB, respectively), and PRRSV ORF7 in between cauliflower mosaic virus (CaMV) 35S constitutive promoter (p35S) and terminator (t35S). The restriction enzyme, HindIII, providing a unique cut within the cassette was used for Southern blot analysis.

FIG. 2 depicts an illustrative embodiment of the anti-PRRSV-ORF7 serum and intestinal antibody titers. Three weekly 10 μg-doses of pure PRRSV-ORF7, soybean-based PRRSV ORF7 (sORF7), or untransformed wild-type soybean (WT) were administered to Balb/c mice by the intragastric route. Anti-PRRSV-ORF7 IgG and IgA antibody titers were determined by ELISA at day 21. Values correspond to mean±SD of titers determined by end-point dilution ELISA in each group of mice (n=5).

FIG. 3 depicts an illustrative embodiment of the anti-PRRSV-ORF7 IgG1 and IgG2a antibody titers. Three weekly doses of 10 μg of pure PRRSV-ORF7, soybean-based PRRSV-ORF7 (sORF7), or untransformed wild-type soybean (WT) were administered to Balb/c mice by the intragastric route. Anti-PRRSV-ORF7 IgG1 and IgG2a antibody titers were determined by ELISA at day 21 in serum samples. Values correspond to means±SD of titers determined by end-point dilution ELISA in each group of mice (n=5).

FIG. 4 depicts anti-PRRSV-ORF7 antibody titers of pigs injected with soybean-based PRRSV-ORF7 as compared to pigs injected with INGELVAC PRRS MLV vaccine. PI, pre-immune. Numbers above bars are P-values.

### DETAILED DESCRIPTION OF THE INVENTION

Porcine reproductive and respiratory syndrome (PRRS), caused by the PRRS virus (PRRSV), is a serious disease of swine and contributes to severe worldwide economic losses in swine production. Current vaccines against PRRSV rely on the use of an attenuated-live virus; however, these vaccines are unreliable. Thus, alternative effective vaccines against PRRSV are needed. Plant-based subunit vaccines offer viable, safe, and environmentally friendly alternatives to conventional vaccines. The PRRSV genome is composed of nine open reading frames (ORFs) (Darwich, et al. (2011) Vet. Microbiol. 150:49-62). ORF1a and 1b encode the viral replicase as well as other non-structural proteins (nsp). Although more than 75% of the viral genome belongs to ORFs 1a and 1b (Dea, et al. (2000) Arch. Virol. 145:659-688), ORFs 2 to 7 play major roles as structural proteins in inducing immune responses. However, the major structural proteins include the envelope glycoprotein GP5 (25 kDa), an unglycosylated M membrane protein (18-19 kDa), and a nucleocapsid (N) protein (15 kDa), encoded by ORFs 5, 6 and 7, respectively. The N protein is an abundant protein of the virion, and four to five domains of antigenic importance have been identified for the N protein (see U.S. Pat. No. 6,495,138). In addition, the N protein has provided consistent induction of antibodies observed in ORF7-vaccinated pigs following three vaccinations (Barfoed, et al. (2004) Vaccine 22:3628-3641).

It has now been shown that ORF7 of PRRSV can be introduced into soybean, Glycine max (L.) Merrill. cvs. Jack and Kunitz, and that the immunogenic protein is properly translated and expressed in soybean seed. The amount of the immunogenic protein accumulating in seeds of these transgenic lines was up to 4.6% of the total soluble protein (TSP). A significant induction of a specific immune response, both humoral and mucosal, against PRRSV-ORF7 was observed following intragastric immunization of BALB/c mice with transgenic soybean seeds. Moreover, intramuscular injection of pigs with transgenic soybean extract containing PRRSV ORF7 protein induced measurable ORF7 antibody responses as early as one week after injection.

Therefore, the present invention is a transgenic soybean expressing, from an exogenous nucleic acid molecule, PRRSV ORF7 immunogen and methods for using the same to induce an antibody response prevent a PRRSV infection. “Transformed,” “transgenic,” “transfected” and “recombinant” refer to a host organism into which an exogenous or heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny, seeds and plant parts such as leaves, roots, and stems. A “non-transformed,” “non-transgenic,” or “non-recombinant” host refers to a wild-type organism, e.g., a plant, which does not contain the heterologous nucleic acid molecule.

As used herein, an “immunogen” is a non-self substance that elicits a humoral and/or cellular immune response in healthy animals such that the animal is protected against future exposure to a pathogen bearing the immunogen. In accordance with the present invention, the immunogen is PRRSV ORF7. As is known in the art, ORF7 encodes the nucleoprotein (N) protein of PRRSV. In one embodiment of this invention, the nucleic acid molecule encoding the PRRSV ORF7 immunogen is isolated from a modified, live, attenuated PRRSV. In particular embodiments, the PRRSV ORF7 immunogen has an amino acid sequence: MPNNNGKQQKRKKGDGQPVNQLCQMLGKIIAQQNQSRGKGPGKKNKKKN PEKPHFPLATEDDVRHHFTPSERQLCLSSIQTAFNQGAGTCTLSDSGRISYTVEFSLPT HHTVRLIRVTASPSA (SEQ ID NO:1). In other embodiments, the PRRSV ORF7 immunogen is a polymorph or genetic variant of SEQ ID NO:1. Examples of genetic variants of SEQ ID NO:1 are provided in Table 1.

TABLE 1

| | SEQ ID |
|---|---|
| ORF7 Sequence* | NO: |
| MPNNNGKQQKRKKGDGQPVNQLCQMLGKTIAQQNQSRGKGPGKKNKKKNPEK | 2 |
| PHFPLATEDDVRHHFTPSERQLCLSSIQTAFNQGAGTCTLSDSGRISYTVEF | |
| SLPTHHTVRLIRVTASPSA | |
| MPNNNGKQQKRKKGDGQPVNQLCQMLGKIIAQQNQSRGKGPGKKNKKKNPEK | 3 |
| PDFPLATEDDVRHHFTRSERQLCLSSIQTAFNQGAGTCTLSDSGRISYTVEF | |
| SLPTHHTVRLIRVTASPSA | |
| MPNNNGKQRKKKKGNGQPVNQLCQMLGKIIAQQNQSRGKGPGKKNKKKSPEK | 4 |
| PDFPLATEDDVRHHFTRSERQLCLSSIQTAFNQGAGTCTLSDSGRISYTVEF | |
| SLPTHHTVRLIRVTASPSA | |
| MPNNNGKQQKKKKGNGQPVNQLCQMLGKIIAQQNQSRGKGPGKKRKKKNPEK | 5 |
| PHFPLATEDDVRHHFTPSERQLCLSSIQTAFNQGAGTCALSDSGRISYTVEF | |
| SLPTQHTVRLIRATASPSA | |

*Hao, et al. (2011) Virol. J. 8: 73.

The nucleic acid molecule encoding PRRSV ORF7 is exogenous to the soybean in the sense that it is foreign or not naturally present in the soybean. However, when expressed in soybean, the PRRSV ORF has now been found to produce a protective immune response upon administration to an animal. As used herein, “immune response” refers to a response made by the immune system of an organism to an immunogen. There are three general types of immune responses including, but not limited to mucosal, humoral and cellular immune responses. A humoral immune response includes the production of antibodies in response to an antigen or antigens. A cellular immune response includes responses such as a helper T-cell (CD4⁺) response and a cytotoxic T-cell lymphocyte (CD8⁺) response. A mucosal immune response results from the production of secretory IgA (sIgA) antibodies in secretions that bathe all mucosal surfaces of the respiratory tract, gastrointestinal tract and the genitourinary tract and in secretions from all secretory glands (McGhee, et al. (1983) Annals NY Acad. Sci. 409). These sIgA antibodies act to prevent colonization of pathogens on a mucosal surface (Williams, et al. (1972) Science 177:697; McNabb, et al. (1981) Ann. Rev. Microbiol. 35:477) and thus act as a first line of defense to prevent colonization or invasion through a mucosal surface. The production of sIgA can be stimulated either by local immunization of the secretory gland or tissue or by presentation of an antigen to either the gut-associated lymphoid tissue (GALT or Peyer's patches) or the bronchial-associated lymphoid tissue (BALT; Cebra, et al. (1976) Cold Spring Harbor Symp. Quant. Biol. 41:210; Bienenstock (1978) Adv. Exp. Med. Biol. 107:53; Weisz-Carrington, et al. (1979) J. Immunol. 123:1705; McCaughan, et al. (1983) Internal Rev. Physiol. 28:131). Membranous microfold cells, otherwise known as M cells, cover the surface of the GALT and BALT and may be associated with other secretory mucosal surfaces. M cells act to sample antigens from the luminal space adjacent to the mucosal surface and transfer such antigens to antigen-presenting cells (dendritic cells and macrophages), which in turn present the antigen to a T lymphocyte (in the case of T-dependent antigens), which process the antigen for presentation to a committed B cell. B cells are then stimulated to proliferate, migrate and ultimately be transformed into an antibody-secreting plasma cell producing IgA against the presented antigen. When the antigen is taken up by M cells overlying the GALT and BALT, a generalized mucosal immunity results with sIgA against the antigen being produced by all secretory tissues in the body (Cebra, et al. (1976) supra; Bienenstock, et al. (1978) supra; Weinz-Carrington, et al. (1979) supra; McCaughan, et al. (1983) supra). Oral immunization is therefore an important route to stimulate a generalized mucosal immune response and, in addition, leads to local stimulation of a secretory immune response in the oral cavity and in the gastrointestinal tract.

An immune response can be measured using techniques known to those of skill in the art. For example, serum, blood or other secretions may be obtained from an organism for which an immune response is suspected to be present, and assayed for the presence of the above mentioned immunoglobulins using an enzyme-linked immunoabsorbant assay (ELISA; Ausubel, et al. (1995) Short Protocols in Molecular Biology, 3^{rd} Ed., John Wiley & Sons, Inc.). According to the present invention, an ORF7 immunogen can be said to stimulate an immune response if the quantitative measure of immunoglobulins in an animal treated with the ORF7 immunogen is, e.g., 2, 3, 4, 5, 10, 20, 30, 40, 50, 60, 70, 80, 90, 100, or 1000-fold or more increased. An increase also means at least 5% or more antibody production, for example, 5, 6, 10, 20, 30, 40, 50, 60 70, 80, 90 or 100% from the measure of immunoglobulins detected in an animal not treated with the ORF7 immunogen, wherein said immunoglobulins are specific for the ORF7 immunogen. A statistical test known in the art and useful to determining the difference in measured immunoglobulin levels includes, but is not limited to ANOVA, Student's T-test, and the like, wherein the P value is at least <0.1, <0.05, <0.01, <0.005, <0.001, and even <0.0001.

An immune response can also be measured using other techniques such as immunohistochemistry using labeled antibodies which are specific for portions of the immunoglobulins raised during the immune response. Tissue from an animal to which the ORF7 immunogen has been administered may be obtained and processed for immunohistochemistry using techniques well-known in the art (Ausubel, et al. (1995) supra). Microscopic data obtained by immunohistochemistry may be quantitated by scanning the immunohistochemically stained tissue sample and quantitating the level of staining using a computer software program known to those of skill in the art including, but not limited to NIH Image (National Institutes of Health, Bethesda, Md.). According to the present invention, the ORF7 immunogen can be said to stimulate an immune response if the quantitative measure of immunohistochemical staining in an animal treated with the ORF7 immunogen is statistically different from the measure of immunohistochemical staining detected in an animal not treated with the ORF7 immunogen, wherein said histochemical staining requires binding specific for the ORF7 immunogen. A statistical test known in the art may be used to determine the difference in measured immunohistochemical staining levels including, but not limited to ANOVA, Student's T-test, and the like, wherein the P value is at least <0.1, <0.05, <0.01, <0.005, <0.001, and even <0.0001.

A transgenic soybean plant of the present invention can be produced by isolating the nucleic acid molecule encoding PRRSV ORF7, inserting said nucleic acid molecule into an expression construct, introducing the expression construct into Agrobacterium tumifaciens, and transforming a soybean with the A. tumifaciens. In one embodiment, the nucleic acid molecule encoding the PRRSV ORF7 immunogen is modified for expression in soybean. In accordance with this embodiment, one or more of the codons of the nucleic acid molecule encoding the PRRSV ORF7 immunogen are mutated (codon optimized) to facilitate expression in soybean. In general, the expression construct of the present invention include in the 5′->3′ direction, a promoter sequence, the nucleic acid sequence encoding the PRRSV ORF7 immunogen; and a termination sequence. The construct may also include selectable marker gene(s), e.g., to kanamycin, neomycin, hygromycin, puramycin, phosphinotricin, chlorsulfuron, methotrexate, gentamycin, spectinomycin, imidazolinones and glyphosate, and/or other regulatory elements for expression, e.g., polyadenylation sequences, a ribosome binding sites and enhancer sequences.

As indicated, the expression construct contains at least one promoter that is operable in a plant cell. As used herein, “promoter” refers to a sequence of DNA, usually upstream (5′) (in other words precedes) of the coding region of a structural gene, which controls the expression of the coding region by providing recognition and binding sites for RNA polymerase and other factors which may be required for initiation of transcription. The selection of the promoter may be dependent upon when and where the gene of interest is to be expressed. For example, an inducible or a constitutive promoter can be used.

The term “inducible” as applied to a promoter is well understood by those skilled in the art. In essence, expression under the control of an inducible promoter is “switched on” or increased in response to an applied stimulus (which may be generated within a cell or provided exogenously). The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus. The preferable situation is where the level of expression increases upon application of the relevant stimulus by an amount effective to alter a phenotypic characteristic. A number of inducible promoters are known in the art. One example of an inducible promoter is the ethanol-inducible gene switch disclosed in Caddick, et al (1998) Nature Biotechnology 16:177-180. Depending upon the objective, the promoter may be a chemically-inducible promoter, where application of the chemical induces gene expression, or a chemical-repressible promoter, where application of the chemical represses gene expression. Chemically inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-Ia promoter, which is activated by salicylic acid. Other chemically regulated promoters of interest include steroid-responsive promoters (see, for example, the glucocorticoid-inducible promoter in Schena, et al. (1991) Proc. Natl. Acad. Sci. USA 88:10421-10425 and McNellis, et al. (1998) Plant J. 14(2):247-257) and tetracycline-inducible and tetracycline-repressible promoters (see, for example, Gatz, et al. (1991) Mol. Gen. Genet. 227:229-237, and U.S. Pat. No. 5,814,618 and U.S. Pat. No. 5,789,156), herein incorporated by reference.

Where enhanced expression in particular tissues is desired, tissue-specific promoters can be utilized. Tissue-specific promoters include those described by Yamamoto, et al. (1997) Plant J. 12(2)255-265; Kawamata, et al. (1997) Plant Cell Physiol. 38(7):792-803; Russell, et al. (1997) Transgenic Res. 6(2):157-168; Rinehart, et al. (1996) Plant Physiol. 112(3):1331-1341; Van Camp, et al. (1996) Plant Physiol. 112(2):525-535; Canevascini, et al. (1996) Plant Physiol. 112(2):513-524; Yamamoto, et al. (1994) Plant Cell Physiol. 35(5):113-118; Lam, (1994) Results Probl. Cell Differ. 20:181-196; Orozco, et al. (1993) Plant Mol Biol. 23(6):1129-1138; Matsuoka, et al. (1993) Proc. Natl. Acad. Sci. USA 90(20):9586-9590; and Guevara-Garcia, et al. (1993) Plant J. 4(3):495-505. Specific examples of promoters of seed storage protein genes include promoters for Zma10Kz or Zmag12.

So-called constitutive promoters may also be used in the methods of the present invention. Constitutive promoters include, for example, rice actin promoter (McElroy, et al. (1990) Plant Cell 2:163-171); ubiquitin promoter (Christensen, et al. (1989) Plant Mol. Biol. 72:619-632 and Christensen, et al. (1992) Plant Mol. Biol. 18:615-689); pEMU (Last, et al. (1991) Theor. Appl. Genet. 87:581-588); MAS promoter (Velten, et al. (1984) EMBO J. 3:2723-2730), soybean seed protein glycinin (Gyl) promoter; soybean vegetative storage protein (vsp) promoter; and granule-bound starch synthase (gbss) promoter, and the like. Other constitutive promoters include those in U.S. Pat. No. 5,608,149; U.S. Pat. No. 5,608,144; U.S. Pat. No. 5,604,121; U.S. Pat. No. 5,569,597; U.S. Pat. No. 5,466,785; U.S. Pat. No. 5,399,680; U.S. Pat. No. 5,268,463; and U.S. Pat. No. 5,608,142. In certain embodiments, the promoter is the CaMV 35-S promoter, derived from the Cauliflower Mosaic Virus, which has frequently been used to drive nominally constitutive expression of foreign genes in plants (Odell, et al. (1985) Nature 313:810-812).

Terminator sequences are typically also present in constructs used in the invention. A terminator is contemplated as a DNA sequence at the end of a transcriptional unit which signals termination of transcription. These elements are 3′-non-translated sequences containing polyadenylation signals, which act to cause the addition of polyadenylate sequences to the 3′ end of primary transcripts. For expression in plant cells the nopaline synthase transcriptional terminator (Depicker, et al. (1982) J. Mol. Applied Gen. 1:561-573) sequence can serve as a transcriptional termination signal.

Plants can be transformed with the expression construct of the invention using standard techniques, which are already known for the genetic manipulation of plants. For example, particle or micro projectile bombardment (U.S. Pat. No. 5,100,792, EP-A-444882, EP-A-434616), microinjection (WO 92/09696, WO 94/00583, EP 331083, EP 175966), electroporation (EP 290395, WO 87/06614), direct DNA uptake (DE 4005152, WO 90/12096, U.S. Pat. No. 4,684,611), liposome-mediated DNA uptake (e.g., Freeman, et al. (1984) Plant Cell Physiol. 29:1353), or the vortexing method (e.g., Kindle (1990) Proc. Natl. Acad. Sci. USA 87:1228) can be used. In certain embodiments, Agrobacterium-mediated transformation is used, wherein PRRSV ORF7 immunogen sequence is flanked by T-DNA left and right border repeats (EP-A-270355; EP-A-0116718; Peralta & Ream (1985) Proc. Natl. Acad. Sci. USA 82:5112-6).

In particular embodiments, the expression construct include a hygromycin resistance gene; Agrobacterium tumifaciens T-DNA left and right border repeats; and a cauliflower mosaic virus 35S constitutive promoter and terminator flanking the PRRSV ORF7 nucleic acid molecule.

Once the expression construct is introduced into plant cells using techniques well-known to those skilled in the art, transformed plant cells can be identified based upon expression of the selectable marker (e.g., hygromycin resistance). Following transformation, a plant may be regenerated, e.g., from single cells, callus tissue, pre-embryogenic masses, embryos, or leaf discs, as is standard in the art. Almost any plant can be entirely regenerated from cells, tissues and organs of the plant. Available techniques are reviewed in Vasil, et al. (1984) Cell Culture and Somatic Cell Genetics of Plants, Vol. I, II and III, Laboratory Procedures and Their Applications, Academic Press; and Weiss Bach & Weiss Bach (1989) Methods for Plant Molecular Biology, Academic Press.

The invention further encompasses a host cell transformed with expression constructs as set forth above, especially a soybean plant cell. In addition, the invention also provides a seed produced by the transgenic plant of the invention. In certain embodiments, the exogenous PRRSV ORF7 immunogen can be expressed at a level of at least 2%, 2.5%, 3%, 3.5%, 4% or 4.5% of the total soluble protein of the seed of the transgenic plant.

The transgenic plant, plant part, seed, or an extract thereof are of particular use in methods for inducing an antibody response and preventing a PRRSV infection. In accordance with such methods, a subject is orally administered an effective amount of the transgenic soybean of the invention. As used herein, a subject refers to an organism classified within the phylogenetic kingdom Animalia. In some embodiments, a subject refers to a mammal, e.g., a mouse, dog, cat, cow, human, deer, horse, sheep, or poultry. In particular embodiments, a subject refers to an animal of the genus Sus, and includes, but is not limited to domestic pig, wild boar, pot-bellied pig, bushpig, babirusa, and warthog. Subjects benefiting from the methods of the invention include animals at risk of PRRSV infection (e.g., healthy animals brought into an infected population) or neonatal pigs.

As used herein, “an effective amount” is an amount necessary to stimulate an immune response, as defined herein, in subject sufficient for the subject to effectively resist a challenge mounted by a pathogen. For example, in one embodiment, “an effective amount” causes an increase in the amount of antibody that binds to the PRRSV ORF7 immunogen, e.g., serum and/or intestinal antibodies. As used herein, an increase means a 2-fold or more, for example, 2, 3, 4, 5, 10, 20, 30, 40, 50, 60, 70, 80, 90, 100, or 1000-fold or more increase in the amount of antibody produced by the vaccinated subject as compared to an unvaccinated subject. An increase also means at least 5% or more antibody production, for example, 5, 6, 10, 20, 30, 40, 50, 60 70, 80, 90 or 100% or more, by a vaccinated subject as compared to an unvaccinated subject. In particular embodiments, an effective amount of transgenic plant, seed or plant part thereof, expressing PRRSV ORF7 immunogen is an amount that provides at least a 300-fold increase in IgA, IgG1 and IgG2a antibody titers in the subject.

The dosages administered to the subject will be determined by a physician or veterinarian in light of the relevant circumstances including the particular transgenic plant, the amount of PRRSV ORF7 immunogen produced by the plant, and the condition of the human or animal. Dosage and administration are adjusted to provide sufficient levels of the active moiety or to maintain the desired effect. Additional factors which may be taken into account include the severity of the age, weight and gender of the subject; diet, time and frequency of administration, drug combination(s), and reaction sensitivities. Generally, 0.1, 1.0, 1.5, 2.0, 5, 10, or 100 mg/kg of an immunogen will be administered to a large mammal. If desired, co-stimulatory molecules or adjuvants can also be provided before, after, or together with the immunogen. Preferably, the dosage of immunogen is administered in the range of 1 ng to 0.5 mg/kg bodyweight, more preferably, 1 mg to 50 mg/kg of body weight.

Efficacy of PRRSV ORF7 immunogen expressed by the transgenic plant, seed or plant part thereof, also referred to herein as an edible vaccine, can be determined by demonstrating that the administration of the vaccine prevents or ameliorates the symptoms of the disease being treated or caused by the pathogen of interest, by at least 5%, preferably 10-20% and more preferably, 25-100%. In the present case, the edible vaccine preferably reduces reproductive failure in a population of sows; reduces or ameliorates cyanosis of the ear and/or vulva; ameliorates respiratory distress in neonatal pigs; and/or decreases susceptibility to secondary infections such as Streptococcus suis, Toxic (O157:H7) Escherichia coli, Salmonella choleraesuis, Haemophilus parasuis, Mycoplasma hyopneumoniae, porcine respiratory coronavirus, swine influenza virus, porcine parvovirus and swine vesicular disease.

The edible vaccine of the invention can be provided as raw plant material, e.g., seeds or plant parts, or alternatively, the transgenic soybean plant, seed or plant part thereof, can be a component of a food product. The term “food product” is intended to include plant material which has been processed, e.g., extracted or ground, and may include additional ingredients, vitamins and/or minerals for eventual ingestion. Food products of the invention include, but are not limited to animal feed, flour, crackers, milk, and the like.

While certain embodiments include the use of edible vaccines, this invention also includes the use of PRRSV ORF7 immunogen that has been isolated from the transgenic plant of the invention. “Isolated” is meant to include plant extracts that are substantially enriched for the PRRSV ORF7 immunogen and/or in which the PRRSV ORF7 immunogen is partially or substantially purified. Partially or substantially purified PRRSV ORF7 immunogen is at least 60% free, preferably 75% free, and most preferably 90% free from other associated components.

Partially or substantially purified PRRSV ORF7 immunogen can be obtained by growing a transgenic soybean plant of the invention for a time sufficient to produce seeds containing the PRRSV ORF7 protein; harvesting the seeds from the plant, wherein the PRRSV ORF7 protein constitutes at least 2.0% of the total soluble protein in the harvested seeds; and purifying the PRRSV ORF7 protein from the harvested seeds. The PRRSV ORF7 protein can be purified by conventional methods including, but not limited to, precipitation, centrifugation, filtration, ultrafiltration, selective digestion, extraction, chromatography, or electrophoresis to obtain the partially or substantially purified PRRSV ORF7 immunogen.

The invention is described in greater detail by the following non-limiting examples.

### EXAMPLE 1

### Materials and Methods

Plant Material. Seeds of soybean [Glycine max (L.) Merr.] cultivars Jack and Kunitz were planted in plastic flats, and grown in the greenhouse. Young pods containing immature cotyledons (5-8 mm in length) were collected, and used as explants for plant transformation.

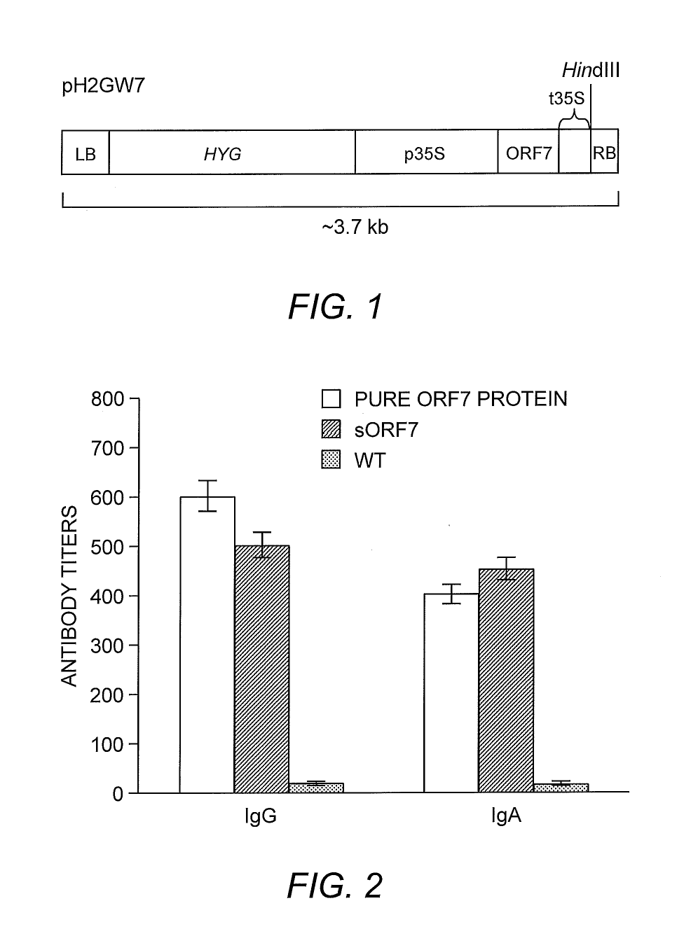

Gene and Vector Constructs. The amino acid sequence of ORF7 (GENBANK Accession NO. U87392; MPNNNGKQQKRKKGDGQPVNQLCQMLGKIIAQQNQSRGKGPGKKNKKKNPEKPHFPLAT EDDVRHHFTPSERQLCLSSIQTAFNQGAGTCTLSDSGRISYTVEFSLPTHHTVRLIRVT ASPSA; SEQ ID NO:1) of a modified live PRRSV (attenuated) vaccine (Boehringer Ingelheim Animal Health, Germany) (Yang, et al. (1998) Arch. Virol. 143:601-612) was reverse-translated, and the synthesized nucleotide sequence was modified for expression in soybean (GENEART Inc., Toronto, ON, Canada). The synthesized ORF7 gene sequence was flanked by unique recombination sites (attB1/attB2), compatible with GATEWAY cloning technology (Invitrogen, California, USA), and cloned into the binary vector pH2GW7 via the entry vector pDONR221. The gene sequence, designated sORF7, was placed between the promoter and the terminator sequences of the cauliflower mosaic virus (CaMV) 35S transcript. The binary vector pH2GW7 also contained spectinomycin (SpR) and hygromycin (Hyg) resistance genes, for plasmid and plant selection, respectively (Karimi, et al. (2005) Trends Plant Sci. 7:193-195) (FIG. 1). The sequence of the plasmid was verified by DNA sequence analysis.

A positive clone was electroporated into Agrobacterium tumefaciens strain KYRT1 following standard protocols. One of the recombinant colonies identified by PCR was grown at 28° C. in Luria-Bertani medium containing 100 mg/L spectinomycin.

Plant Transformation. Young pods were surface-sterilized with 70% 2-isopropanol and 25% CLOROX (6% sodium hypochlorite), and then rinsed three times with sterilized deionized water. After removing seed coats and embryonic axes, disinfected cotyledons were cocultivated with a bacterial culture of Agrobacterium carrying the construct, and the transformation was conducted following protocols described previously, but with a slight modification (Ko, et al. (2003) Theor. Appl. Genet. 107:439-447; Ko, et al. (2004) Crop Sci. 44:1825-1831). Somatic embryos (SEs) were induced on a selection medium composed of Murashige and Skoog (MS) medium (Murashige & Skoog (1962) Physiol. Plant 473-97) and Gamborg's B5 vitamins along with 40 mg/L 2,4-dichlorophenoxyacetic acid (2,4-D), 3 g/l sucrose, and containing 10 mg/L hygromycin B along with 500 mg/l cefotaxime. The medium was solidified with 2 g/L GELRITE gellan gum before autoclaving. The pH of the medium was adjusted to 5.7 with 1 N KOH. After 28 days on the selection medium, induced SEs were excised from responding explants, and directly placed on a maturation medium (MM), composed of MS salts, Gamborg's B5 vitamins, 6 g/L maltose, 500 mg/L cefotaxime, and supplemented with 25 mg/L hygromycin B for an additional 7 weeks. Cultures were transferred to fresh medium biweekly to promote maturation of SEs. Mature SEs were then placed on a regeneration medium (RM) composed of MS salts, Gamborg's B5 vitamins, 25 mg/L hygromycin, 500 mg/L cefotaxime, and 30 g/L sucrose until both shoots and roots were observed. Young plantlets were transferred to pots containing sterile Sunshine soil mix, and grown in the greenhouse for flowering and pod set.

Molecular Analysis. A DNase DNA kit (Qiagen, San Diego, Calif., USA) was used to extract genomic DNA (gDNA) from 100 mg of leaf tissue for PCR analysis. The concentration of extracted DNA was determined using a NANODROP ND-1000 UV-Vis Spectrophotometer (NANODROP Technologies, Delaware, USA). Approximately 100 ng of sample genomic DNA was amplified by PCR using primers corresponding to the introduced transgene (ORF7F-5′-GCGGCCGCACTAGTGATA-3′ (SEQ ID NO:6), ORF7R-5′-GATGGAGAAGCGGTAACCC-3′ (SEQ ID NO:7)). Reactions were denatured at 94° C. for 3 minutes, followed by 33 cycles of denaturation (94° C. for 30 seconds), annealing (65° C. for 30 seconds), extension (72° C. for 1 minute), and a final extension (72° C. for 10 minutes). PCR products were run on 1% agarose gels containing ethidium bromide, and visualized by UV exposure using a KODAK imaging system (KODAK Image station 2000R, Carestream Health Molecular Imaging, Connecticut, USA).

For Southern blot analysis, extraction of large amounts of DNA was conducted in accordance with known methods (Dellaporta, et al. (1983) Plant Mol. Biol. Rep. 1:19-21) with some modifications, i.e., chloroform:isoamyl alcohol (24:1) were added, the sample was centrifuged for 10 minutes at 4 C, the supernatant was then transferred to a new tube, DNA was precipitated by isopropanol, and the pellet with then washed with 70% ethanol. Genomic DNA was digested overnight with HindIII and subjected to electrophoresis on a 0.8% agarose gel. A PCR-DIG probe synthesis kit (Roche, Indianapolis, Ind., USA) was used for probe labeling, and DIG Easy Hyb (Roche) was used for hybridization and detection. The synthesis of a PCR-labeled probe was evaluated by agarose gel electrophoresis. DNA on the gel was transferred onto a nitrocellulose membrane (HYBOND-N+; Amersham Biosciences, New Jersey, USA) using the capillary transfer method and probed with a digoxigenin (DIG)-labeled ORF7 probe corresponding to the ORF7 transgene, according to the manufacturer's guidelines. This was followed by hybridization and detection, also following the manufacturer's guidelines. The membrane was then washed and visualized on a chemiluminescense film (HYBLOT CL; Denville Scientific, Inc., New Jersey, USA).

Reverse Transcriptase-PCR Analysis. To investigate expression levels of the transgene, reverse transcriptase-PCR (RT-PCR) was conducted. Total RNA was extracted from approximately 100 mg of T₀ leaf tissues using RNAQUEOUS (Ambion, Texas, USA), according to the manufacturer's guidelines. The SUPERSCRIPT First-Strand synthesis kit (Invitrogen) was used for reverse transcription. The complementary DNA was synthesized from DNAse-treated RNA by SUPERSCRIPT II RT (Invitrogen) following the addition of RNase H to remove RNA, according to manufacturer's guidelines. Subsequently, 1 μl of RT reaction was used for PCR. The target cDNA was amplified by PCR using PRRSV-ORF7 primers, described above, and tubulin primers (tubB2F-5′-GTGACTTGAACCATCTGATCTCAGC-3′ (SEQ ID NO:8) and tubB2R-5′-GTTGAAGCCATCCTCAAGCCAG-3′ (SEQ ID NO:9)) as described in the art (Valer, et al. (2006) FEMS Microbiol. Lett. 265:60-68). The tubulin gene was used as a constitutive control. The PCR program included incubation at 94° C. for 3 minutes, followed by 33 cycles at 94° C. for 30 seconds, 65° C. for 30 seconds, 72° C. for 1 minute, and a final extension at 72° C. for 10 minutes. Amplified products were analyzed by agarose gel electrophoresis, and visualized by UV exposure using the KODAK Image station 2000R.

Protein Analysis. Total soluble protein (TSP) of approximately 100 mg of ground soybean seeds was extracted using 1 ml extraction buffer (50 mM Tris-HCl (pH 7.5) and 10 μl/ml protease inhibitor cocktail (Sigma), and centrifuged at 14,000 rpm (EPPENDORF centrifuge 5417R) at 4° C. for 1 hour. The total amount of soluble protein was quantified using the Bradford reagent (Bio-Rad) against a BSA standard.

For western blot analysis, SDS-PAGE was performed. A total of 25 μg of TSP was loaded onto 4-20% acrylamide iGels (NuSep, Georgia, USA). The gels were blotted onto BIOTRACE polyvinylidene fluoride (PVDF) membrane (Pall Corporation, New York, USA). After blocking with 5% nonfat dried milk, the blots were incubated overnight with 1:100 dilution of a rabbit anti-PRRSV-ORF7 polyclonal antibody (Open Biosystems, Alabama, USA) following by a 1 hour incubation with goat anti-rabbit peroxidase (1:10000 dilution; Sigma-Aldrich, Missouri, USA). The Lumi-Light Western Blotting Substrate (Roche) was used for detection, following the manufacturer's instructions. Image analysis was conducted using the KODAK Imaging Station 2000R.

Mice Immunization and Immunogenicity Assessment. Eight to 10-week-old female BALB/c mice (Harlan Sprague Dawley, Indiana, USA) were used. All animals were handled according to federal regulations for animal experimentation and care (NOM-062-ZOO-1999, Ministry of Agriculture, Mexico), and approved by the Institutional Animal Care and Use Committee.

Mice were immunized via the intragastric route with 60 mg of ground soybean seed (powder) containing 10 μg of soybean-derived sORF7. Positive and negative controls included pure ORF7 protein (10 μg) and 60 mg of wild-type soybean seeds, respectively. Each test group contained five animals for which three oral doses were administered on days 0, 7, and 14. Soybean seed powder was resuspended in 0.5 ml water, and was administered to test animals via the intragastric route. Animals were sacrificed on day 21 to collect serum samples and intestinal fluids.

Anti-PRRSV-ORF7 Antibody ELISA Assays From Immunized Mice. Serum samples were obtained from blood extracted following a cardiac puncture of ether-anesthetized mice. Intestines were harvested and flushed with 5 mL of cold RPMI 1640 medium. Subsequently, 500 μL of 10 mM p-hydroxymercuribenzoate (dissolved in 150 mM Tris-base) was added. Samples were centrifuged at 4° C. for 10 minutes at 12,000 g, supernatants were collected, and stored at −70° C. until needed for assay.

Antibody contents in serum and intestinal fluids were determined by an indirect enzyme-linked immunosorbent assay (ELISA). A volume of 50 μL was used for all assays. Briefly, 96-well plates were coated with ORF7 pure protein (0.01 μg/μL) diluted in bicarbonate-carbonate buffer (15 mM Na₂CO₃, 35 mM NaHCO₃, pH 9.6) for 2 hours at 37° C. Plates were washed, and then blocked with 5% nonfat dry milk dissolved in phosphate-buffered saline (PBS; 100 mM NaCl, 10 mM Na₂HPO₄, 3 mM KH₂PO₄, pH 7.2). Two-fold dilutions of serum samples (beginning with 1:100) were diluted 1:10 in PBST (0.05% v/v TWEEN-20 in PBS); while intestinal fluid samples were diluted 1:50 with 5% non-fat milk dissolved in PBST. Samples were added to sensitized plates, and incubated overnight at 4° C. Labeling was done using goat anti-mouse IgG, IgA, IgG1, or IgG2a horseradish peroxidase conjugates (Zymed Laboratories, California, USA). Plates were incubated for 1 hour at 37° C. and then washed with PBST. Subsequently, plates were incubated at room temperature along with a substrate solution (0.5 mg/ml o-phenylenodiamine, 0.01% H₂O₂, 50 mM citrate buffer, pH 5.2). Following color development (15 minutes), the reaction was stopped with 25 μl of 2.5 M H₂SO₄.

Titers were recorded as reciprocal values of the highest end-point dilution of samples having OD₄₉₂>0.100. ELISA data corresponded to the geometric means of n=5 mice per group, and were representative of duplicate experiments. The comparisons between groups were made using Tukey's test (P value<0.05).

Pig Immunization and Immunogenicity Assessment. At 2 weeks of age, blood was collected from pigs and serum was validated PRRSV-free by RT-PCR. At 3 weeks of age, nine pigs, representing three litters, were randomly assigned to three vaccine groups with one pig from each litter per group. On day 0, all pigs were injected intramuscularly with a dose of their respective vaccine: transgenic soybean extract expressing 1 mg PRRSV ORF7 protein+adjuvant (EMULSIGEN-D; which contains dimethyldioctadecyl ammonium bromide (DDA) for added immune stimulation); or INGELVAC PRRS MLV vaccine (Boehringer Ingelheim, St. Joseph, Mo.). On day 21, all pigs were given a second dose of their respective vaccine. All pigs were bled on days 7, 14, 21, 28, 35, and 42. The sera were processed and stored at −80° C. until analyzed for PRRSV ORF7 antibodies.

Anti-PRRSV-ORF7 Antibody ELISA Assays From Immunized Pigs. NUNC-IMMUNO MAXISORP 96 well-flat-bottom plates were coated with 50 μL/well of 10 μg/mL PRRSV ORF7 peptide (prepared in carbonate-bicarbonate buffer) and allowed to incubate overnight at 4° C. After four washes with PBS-TWEEN 20 buffer (pH 7.4), wells were blocked with 200 μL/well of 3% BSA blocking buffer prepared in PBS-TWEEN 20 and incubated for 1.5 hours at room temperature. Liquid was removed, but the plates were not washed prior to serum addition. Based on the results of antibody titration assays, all sera (pre-immune and vaccinated) were diluted 1:20 with 3% BSA blocking buffer. Seventy-five microliters of the prepared serum samples were added to each well with four wells per sample. Plates were incubated at room temperature for 1.5 hours followed by four washes with PBS-TWEEN 20 buffer. After removal of the wash solution, 50 μL of Rabbit-Anti Porcine IgG was added to each well. Plates were incubated at room temperature for 1.5 hours followed by washing as described above. Subsequently, 200 μL of pNPP substrate solution was dispensed into each well. Plates were incubated at room temperature for 30 minutes after which 50 μL of 3N NaOH stop solution was added to each well to stop color reaction. The absorbance at 405 nm was measured and recorded.

### EXAMPLE 2

### Soybean Transformation and ORF7 Expression

The nucleotide sequence corresponding to ORF7 was derived from the amino acid sequence of a modified live PRRSV vaccine (Yang, et al. (1998) supra). The nucleotide sequence was modified to provide a GC content suitable for protein expression in soybean. The modified nucleotide sequence was composed of 437 nucleotides encoding 123 amino acid sequences. The predicted molecular mass of the PRRSV-ORF7 protein was 14 kDa.

To express the PRRSV-ORF7 protein in soybean, the GATEWAY expression vector, pH2GW7 plasmid, harboring the PRRSV-ORF7 gene construct, was used to transform immature cotyledons of soybean cvs. Jack and Kunitz. A schematic diagram of the gene construct is presented (FIG. 1).

Following Agrobacterium-mediated transformation, somatic embryos (SEs) were observed along the edges of explants. SEs underwent the typical histological differentiation from globular, heart, torpedo, and cotyledonary stages of development. Their maturity was observed when SEs at the cotyledonary stage turned yellowish green at which they were desiccated prior to conversion into whole plantlets.

Hygromycin-resistant somatic embryos (Hyg^{R} SEs), carrying the PRRSV-ORF7 transgene, were successfully induced from explants of both soybean cultivars. Although several induced SEs were observed at the globular stage, most failed to convert into whole plantlets. However, some SEs carrying PRRSV-ORF7 regenerated into whole plantlets, and these were then acclimatized, and transferred to the greenhouse. Eight independent putative transgenic lines, five lines from cv. Jack, designated TJ1, TJ2, TJ3, TJ4, and TJ5, and three lines from cv. Kunitz, designated TK1, TK2, and TK3, were obtained, and these were grown to maturity in the greenhouse.

Presence of the PRRSV-ORF7 transgene in eight putative lines was initially confirmed by PCR. Genomic DNA (gDNA) was isolated from T₀ leaf tissues, and a set of primers flanking the PRRSV-ORF7 transgene was used in PCR reactions. Amplification of the PRRSV-ORF7 should yield a product of 437 by in size. The plasmid DNA harboring the PRRSV-ORF7 transgene and gDNA of untransformed soybean cvs. Jack and Kunitz were isolated, and served as positive and negative controls, respectively. Out of eight putative transgenic lines, a single transgenic plant (TJ1) from cv. Jack and three transgenic plants (TK1, TK2, and TK3) from cv. Kunitz showed a 437-bp band corresponding to the PRRSV-ORF7 transgene. Those lines that did not reveal the PCR product for PRRSV-ORF7 were deemed non-transformed plants (escapes), and were discarded.

To further confirm the integration of the transgene into the soybean genome, Southern blot analysis was conducted. The gDNA from T₀ leaf was isolated and digested with HindIII, as it was a unique restriction site within the T-DNA insert, and it was probed with a labeled PRRSV-ORF7. The presence of tandem T-DNA copies and the total number of T-DNA loci within a line were identified in each of the four PCR-positive lines. The sizes of restricted fragments containing the T-DNA insert observed in these lines were -3.7 kb. The HindIII-digested plasmid DNA of PRRSV-ORF7-pH2GW7 and HindIII-digested genomic DNA of untransformed soybean cvs. Jack and Kunitz served as positive and negative controls, respectively. Southern blots revealed three different patterns of hybridization from these four transgenic lines. Either single or multiple copies of the transgene were detected in these four transgenic lines. A single band in transgenic line TJ1 of 5.5 kb in size was detected. Two transgenic lines, line TK1 and TK2, showed identical banding patterns, each containing three bands of 5.5 kb, 4.7 kb, and 3.7 kb in size, therefore they were likely to be from the same transformation event. Whereas, transgenic line TK3 showed presence of two bands of 7.4 kb and 8.3 kb in size.

Thus, all transformed plants derived from cv. Kunitz were confirmed to be transgenic; while, one out of five transformed plants of cv. Jack was confirmed to be transgenic. Overall, the frequencies of transformation (calculated based on number of confirmed transgenic plants per number of co-cultivated explants) for cvs. Jack and Kunitz reached 1.06% and 2.41%, respectively.

Messenger RNA transcription in all four transgenic soybean lines, TJ1, TK1, TK2, and TK3, was determined using reverse transcription PCR (RT-PCR). Presence of a band from all confirmed transgenic lines corresponding to the plasmid DNA harboring the sORF7 transgene, serving as positive control, indicated that the PRRSV-ORF7 transgene was transcribed in each of these lines. Expression of the PRRSV-ORF7 protein was analyzed in seed of T1 positive plants of lines TK1, TK2, and TK3 using western blot analysis against the PRRSV-ORF7 peptide. Total soluble protein (TSP) from these seeds was extracted, and the percentage of the PRRSV-ORF7 peptide in TSP of each sample was calculated densitometrically by western blot using the PRRSV-ORF7 as a standard. All T1 positive plants contained the expected 14 kDa protein of the synthetic PRRSV-ORF7 while no signal was detected in wild-type plants. Two additional bands detected on the gel corresponded to polymers, dimers (˜28 kDa) or trimers (˜40 kDa), of the peptide.

### EXAMPLE 3

### Immunogenicity of Transgenic Soybean-Derived PRRSV-ORF7

Prior to use of TK3 seeds for mice immunization, the TSP was determined in these seeds. The TSP was extracted from 16 grams of soybean seed, and sORF7 content in soybeans seeds was determined to equal 262 μg per gram of ground seed. The dosage per treatment used for mice immunization was 60 mg of soybean powder, containing 10 μg of PRRSV-ORF7. Serum and intestinal antibody responses were determined after administering three intragastric dosages of either PRRSV-ORF7 pure protein, soybean-derived PRRSV-ORF7, or wild-type soybean (FIG. 2). Mice fed with transgenic soybean expressing PRRSV-ORF7 elicited significant serum IgG anti-PRRSV-ORF7 antibody responses similar to those elicited by the PRRSV-ORF7 pure protein. Furthermore, a specific IgA response was detected in the intestinal fluids whose magnitude was similar to that elicited by the pure PRRSV-ORF7 (FIG. 2). These results demonstrated that the soybean-derived PRRSV-ORF7 was immunogenic when administered by the intragastric route.

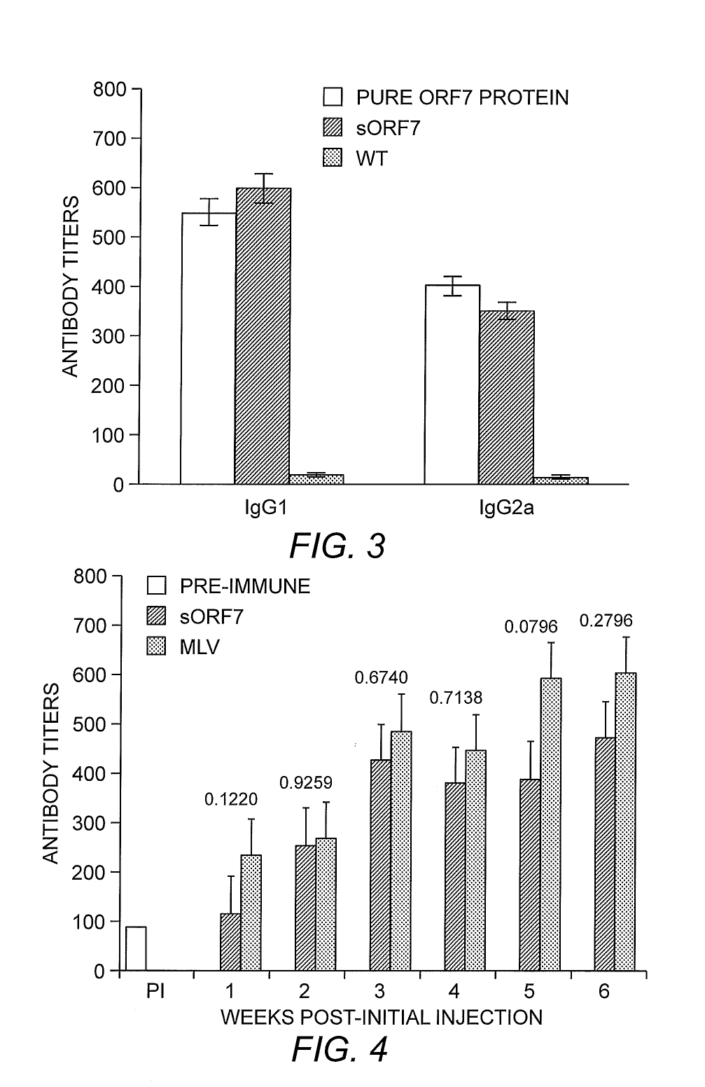

Specific anti-PRRSV-ORF7 serum IgG1 and IgG2a antibodies were elicited in mice immunized with soybean-derived PRRSV-ORF7 (FIG. 3). In so far as higher levels of IgG1 than IgG2a antibody responses were elicited in serum samples (FIG. 3), it was concluded that a humoral immune response was predominantly elicited.

Immunization of young pigs with soybean-derived PRRSV-ORF7 was also carried out. At three weeks of age, pigs were injected with transgenic soybean extract expressing 1 mg PRRSV ORF7 protein+adjuvant or INGELVAC PRRS MLV vaccine. All pigs were given a second dose of their respective vaccine on day 21. All pigs were bled on days 7, 14, 21, 28, 35, and 42 and anti-PRRSV ORF7 antibody titer was determined. This analysis indicated that the soybean-derived PRRSV-ORF7 protein elicited an antibody response comparable to the INGELVAC PRRS MLV vaccine (FIG. 4).

Overall, this analysis indicated that PRRSV-ORF7 can be expressed in soybean at a level up to 4.6% TSP in soybean seed. Compared to the soybean-derived LTB antigen (Moravec, et al. (2007) Vaccine 25:1647-1657), PRRSV-ORF7 levels detected in this study were about two-fold higher. The PRRSV-ORF7 protein expressed in soybean was of the correct molecular mass of the PRRSV ORF7 antigen and detected in seeds of T₁ transgenic soybean cv. Kunitz lines. The immunogenicity of this candidate soybean-based PRRS vaccine was confirmed in immunized mice. Specific humoral and mucosal immune responses were elicited following intragastric administration of three doses of soybean seeds containing 10 μg of PRRSV-ORF7. This was similar to antibody levels detected in mice administered three doses of 10 μg of pure PRRSV-ORF7. Analysis of IgG subclasses revealed that an appropriate Th1 response was predominantly elicited in test mice, which was consistent with the desired protective immune response (Perlman & Dandekar (2005) Nat. Rev. Immunol. 5:917-927). Thus, the sORF7 antigen was properly produced in soybean cellular machinery and conserving its immunogenic properties.

What is claimed is:

1. A transgenic soybean plant comprising an exogenous nucleic acid molecule encoding porcine reproductive and respiratory syndrome virus (PRRSV) ORF7 immunogen, wherein the ORF7 immunogen produces a protective immune response upon administration to an animal.

2. A seed from the transgenic soybean plant of claim 1, wherein at least 2.0% of total soluble protein of the seed is the PRRSV ORF7 immunogen.

3. A plant part from the transgenic soybean plant of claim 1.

4. A food product comprising the transgenic soybean plant of claim 1, or seed or plant part thereof.

5. An expression construct for expressing an immunoprotective immunogen in a soybean plant cell comprising a nucleic acid molecule encoding porcine reproductive and respiratory syndrome virus (PRRSV) ORF7 immunogen.

6. The expression construct of claim 5, wherein the expression construct comprises:
(a) a hygromycin resistance gene;
(b) Agrobacterium tumifaciens T-DNA left and right border repeats; and
(c) a cauliflower mosaic virus 35S constitutive promoter and terminator flanking the PRRSV ORF7 nucleic acid molecule.

7. A transgenic soybean plant cell transformed with the expression construct of claim 5.

8. A method for preparing a porcine reproductive and respiratory syndrome virus (PRRSV) immunogen in soybean comprising:
(a) modifying a PRRSV ORF7 nucleic acid for expression in soybean;
(b) inserting the modified PRRSV ORF7 nucleic acid into an expression construct;
(c) introducing the expression construct of (b) into Agrobacterium tumifaciens; and
(d) transforming soybean with the Agrobacterium tumifaciens thereby preparing a PRRSV immunogen in plants.

9. A transgenic soybean prepared by the method of claim 8.

10. A seed from the transgenic soybean of claim 9.

11. A method for obtaining a purified porcine reproductive and respiratory syndrome virus (PRRSV) ORF7 protein comprising:
(a) growing the transgenic soybean plant of claim 1 for a time sufficient to produce seeds containing the PRRSV ORF7 protein;
(b) harvesting the seeds from the transgenic soybean plant, wherein the PRRSV ORF7 protein constitutes at least 2.0% of the total soluble protein in the harvested seeds; and
(c) purifying the PRRSV ORF7 protein from the harvested seeds.

12. A method for inducing an antibody response comprising orally administering an effective amount of the transgenic soybean plant of claim 1, or seed or plant part thereof, to an animal thereby inducing an antibody response in the animal.

13. A method for preventing a porcine reproductive and respiratory syndrome virus infection comprising orally administering to a subject in need thereof the transgenic soybean plant of claim 1, or seed or plant part thereof, wherein the PRRSV ORF7 immunogen of said transgenic soybean plant, seed or plant part thereof, produces a protective immune response which prevents porcine reproductive and respiratory syndrome virus infection.
